(19) United States
(12) Patent Application Publication
(10) Pub. No.: US 2007/0098678 A1
(43) Pub. Date: May 3, 2007

(54) Enteric coated aliphatic amine polymer bile acid sequestrants

(76) Inventors: Hitesh R. Bhagat, Wayland, MA (US); Joanne M. Donovan, Needham, MA (US); William Braunlin, Arlington, MA (US); Robert Sacchiero, Hudson, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/475,385

(22) Filed: Jun. 26, 2006

Related U.S. Application Data
(63) Continuation of application No. PCT/US04/43537, filed on Dec. 23, 2004.
(60) Provisional application No. 60/533,563, filed on Dec. 31, 2003.

(51) Int. Cl. A61K 31/785; A61K 9/20

(52) U.S. Cl. 424/78.27; 424/464

(57) ABSTRACT

Tablets, capsules, sachets, or papers having one or more aliphatic amine polymers allow for the targeted release of the polymers at a specific region of the gastrointestinal tract, especially the small intestine. These tablets, capsules, sachets, or papers are useful in a method for lowering cholesterol in a mammal in need thereof. The tablet includes a tablet core having an aliphatic amine polymer, and an enteric coating for the core. The capsule, sachet or paper includes a plurality of beads where the beads have a bead core having an aliphatic amine polymer, an enteric coating therefor and optionally a water-soluble coating.

### RELATED APPLICATIONS

This application is a continuation of International Application No. PCT/US2004/043537, which designated the United States and was filed on Dec. 23, 2004, published in English, and which claims the benefit of U.S. Provisional Application No. 60/533,563, filed on Dec. 31, 2003. The entire teachings of these applications are incorporated herein by reference.

### BACKGROUND OF THE INVENTION

A number of aliphatic amine polymers have been described for treatment of various conditions such as hyperlipidemia and hypercholesterolemia. Many of these aliphatic amine polymers function as non-absorbed ion exchange resins in the digestive tract. Such non-absorbed aliphatic amine polymers bind or otherwise sequester bile acids, a metabolic product of cholesterol, and prevent their absorption by circulation through the small intestine and liver. Examples of such bile acid sequestrants (BAS) include a variety of aliphatic amine polymers useful as cholesterol lowering agents, disclosed in U.S. Pat. Nos. 5,607,669, 5,624,963, 5,679,717 and 6,423,754, W098/29107 and WO099/22721.

Therapeutically effective dosages of aliphatic amine polymers for lowering serum cholesterol of a patient are generally large. For example, therapeutically effective dosages of a poly(allylamine hydrochloride) crosslinked with epichorohydrin and alkylated with 1 -bromodecane and (6-bromohexyl)-trimethylammonium bromide (described in U.S. Pat. Nos. 5,607,669 and 5,679,717), also referred to as colesevelam, and marketed in the United States as Welchol™, are typically on the order of 3 to 6 grams per day. Consequently, development of a dosage form of aliphatic amine polymers that can lower the required doses of aliphatic amine polymers would be advantageous.

### SUMMARY OF THE INVENTION

The present invention is directed to tablets, capsules, sachets and papers that have an aliphatic amine polymer-containing core having an enteric coating that targets the release of aliphatic amine polymers to one or more specific intestinal regions. The release of aliphatic amine polymers in particular regions of the intestinal tract will increase the therapeutic effect of the aliphatic amine polymers, thereby reducing the required dose of aliphatic amine polymers.

In one aspect, the invention is generally directed to a tablet that includes a tablet core and a pharmaceutically acceptable enteric coating therefor. The tablet core includes an aliphatic amine polymer. In one embodiment, the enteric coating solubilizes in an aqueous solution between about pH 5.0 and about pH 6.0 at about 37° C. In another embodiment, the enteric coating solubilizes in an aqueous solution between about pH 6.0 and about pH 7.0 at about 37° C.

In another aspect, the invention includes a tablet having a tablet core that includes an aliphatic amine polymer, and a pharmaceutically acceptable enteric coating therefor. When administered orally to a mammal, the tablet releases the aliphatic amine polymer at a specific region of the small intestine, i.e., the duodenum, jejunum or ileum.

The present invention also includes a capsule, sachet or paper having a first plurality of beads having an aliphatic amine polymer and a pharmaceutically acceptable enteric coating therefor, where the enteric coating solubilizes in an aqueous solution in the range of between about pH 5.0 and about pH 7.0 at about 37° C.

In a further aspect, the invention relates to a capsule, sachet or paper having a first plurality of beads that includes an aliphatic amine polymer and a pharmaceutically acceptable enteric coating therefor, where the capsule, sachet or paper, when administered orally to a mammal, releases the aliphatic amine polymers in the duodenum, jejunum or ileum of the mammal at body temperature. The capsule, sachet or paper according to the invention can further include a second plurality of beads having a different enteric coating or a different amount of enteric coating from the first plurality of beads, so that the enteric coating solubilizes at a different pH or the second plurality of beads release the aliphatic amine polymer at a different region of the small intestine of a mammal.

The present invention also relates to a tablet comprising a tablet core having a polymer active ingredient, where the tablet core is coated with a water-soluble coating and the water-soluble coating is coated with an enteric coating.

The present invention further relates to a method for lowering cholesterol in a mammal in need thereof by administering to the mammal a therapeutically effective amount of one or more tablets, capsules, sachets, or papers of the invention.

### BRIEF DESCRIPTION OF THE DRAWINGS

FIG. 1 is a graph showing the percent weight gain of Eudragit® L30D-55 enteric coating (topcoat) versus disintegration time at pH 5.75 for 800 mg Renagel® tablets having a 2.5% sealcoat of hydroxypropylmethyl cellulose.

FIG. 2 is a plot of disintegration time of 800 mg Renagel® tablets versus percent weight gain of Eudragit® L30D-55 enteric coating (topcoat) at pH 5.75 and pH 6.25 in 0.05M succinate buffer.

FIG. 3 is a graph showing the effect of enteric coating (topcoat) weight gain on the disintegration time of 625 mg Welchol™ tablets at pH 5.75.

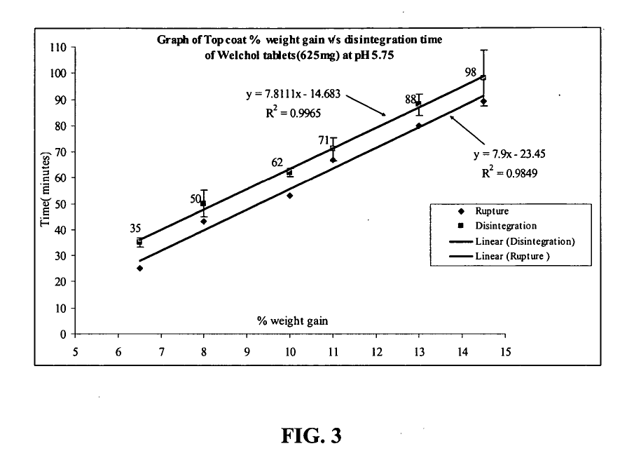

FIG. 4 is a graph showing the effect of sealcoat weight gain on the disintegration time of 625 mg Welchol™ tablets at pH 5.75.

### DETAILED DESCRIPTION OF THE INVENTION

Aliphatic amine polymers generally are known to function as bile acid sequestrants, which lower serum cholesterol levels. For example, a poly(allylamine hydrochloride) crosslinked with epichorohydrin and alkylated with 1-bromodecane and (6-bromohexyl)-trimethylammonium bromide (described in U.S. Pat. Nos. 5,607,669 and 5,679,717, the contents of which are incorporated herein by reference), referred to as colesevelam, and marketed in the United States as Welchol™, has been shown to be effective in lowering the serum cholesterol level of a patient. In another example, an epichorohydrin-cross-linked poly(allylamine hydrochloride) resin (described in U.S. Pat. No. 6,423,754, the contents of which are incorporated herein by reference), referred to as sevelamer, and marketed as Renagel®, has been shown to be effective for treating hypercholesterolemia.

An aliphatic amine polymer used in the invention is a polymer which is manufactured by polymerizing an aliphatic amine monomer. An aliphatic amine is saturated or unsaturated, straight-chained, branched or cyclic non-aromatic hydrocarbon having an amino substituent and optionally one or more additional substituents. The aliphatic amine polymer can be one of the aliphatic amine polymers described in U.S. Pat. Nos. 5,487,888, 5,496,545, 5,607,669, 5,618,530, 5,624,963, 5,667,775, 5,679,717, 5,703,188, 5,702,696, 5,693,675, 5,900,475, 5,925,379, 6,083,497, 6,177,478, 6,083,495, 6,203,785, 6,423,754, 6,509,013 and 6,556,407, and U.S. Published Applications Nos. 2002/0159968 A1, 2003/0086898 A1 and 2003/0133902 A1, the contents of which are incorporated herein by reference in their entireties. Polymers suitable for use in the invention are also disclosed in U.S. application Ser. No. 08/823,699 (now abandoned); Ser. No. 08/835,857 (now abandoned); Ser. No. 08/470,940 (now abandoned); Ser. No. 08/927,247 (now abandoned); Ser. Nos. 08/964,498; 09/691,429 and 10/125,684, the contents of which are incorporated herein by reference in their entireties.

Examples of aliphatic amine polymers include polymers that have one or more repeat units represented by at least one formula from the group consisting of: 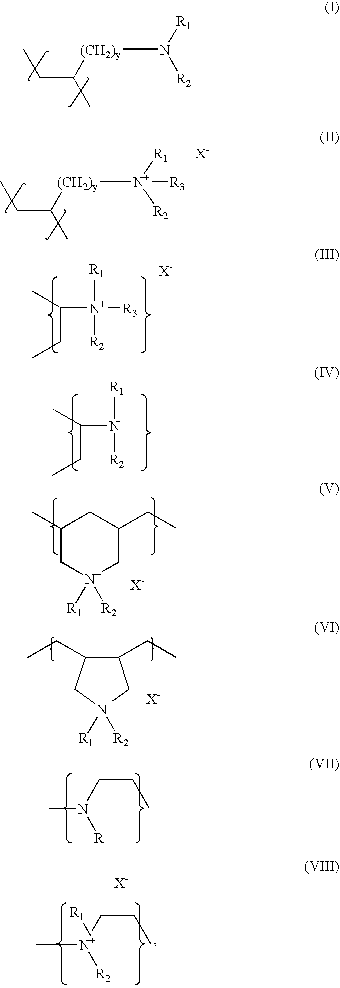
or a salt or copolymer thereof, where y is an integer of one or more (e.g., between about one and about 10, preferably between one and four, more preferably one) and each R, R₁, R₂, and R₃, independently, is H, a substituted or unsubstituted alkyl group (e.g., having between 1 and 25 or between 1 and 5 carbon atoms, inclusive) or aryl (e.g., phenyl) group, and each X⁻ is an exchangeable negatively charged counterion.

In preferred polymers used in the invention, at least one of R, R₁, R₂, or R₃ is a hydrogen atom. More preferably, each of these groups is hydrogen.

As an alkyl or aryl group, R, R₁, R₂, and R₃ can carry one or more substituents. Suitable substituents include cationic groups, e.g., quaternary ammonium groups, or amine groups, e.g., primary, secondary or tertiary alkyl or aryl amines. Examples of other suitable substituents include hydroxy, alkoxy, carboxamide, sulfonamide, halogen, alkyl, aryl, hydrazine, guanidine, urea, poly(alkyleneimine) such as poly(ethylenimine), and carboxylic acid esters.

In a particularly preferred embodiment, the aliphatic amine polymer is a polyallylamine, alkylated polyallylamine, polyvinylamine, poly(diallylamine) or poly(ethyleneimine) or a salt thereof with a pharmaceutically acceptable acid. The aliphatic amine polymer is optionally substituted at one or more nitrogen atoms with an alkyl group or a substituted alkyl group such as a trialkylammonioalkyl group. The aliphatic amine polymer can optionally be cross-linked by means of a multifunctional cross-linking agent, for example via a multifunctional monomer or a bridging group which connects two amino nitrogen atoms from two different polymer strands.

The preferred polymers employed in the invention are water-insoluble, non-absorbable, cross-linked polyamines.

Polymers suitable for use in the invention can be homopolymers or copolymers.

A multi-functional cross-linking agent can be characterized by functional groups which react with the amino group of the monomer or polymer. Alternatively, the cross-linking group can be characterized by two or more vinyl groups which undergo free radical polymerization with the amine monomer. The degree of polymerization in cross-linked polymers (i.e., the value of “n”) cannot generally be determined because of the insolubility and size of these polymers.

Examples of suitable multifunctional cross-linking agents include diacrylates and dimethylacrylates (e.g. ethylene glycol diacrylate, propylene glycol diacrylate, butylene glycol diacrylate, ethylene glycol dimethacrylate, propylene glycol dimethacrylate, butylene glycol dimethacrylate, polyethyleneglycol dimethacrylate and polyethyleneglycol diacrylate), methylene bisacrylamide, methylene bismethacrylamide, ethylene bisacrylamide, ethylene bismethacrylamide, ethylidene bisacrylamide, divinylbenzene, bisphenol A, dimethacrylate and bisphenol A diacrylate. Other examples of suitable multi-functional cross-linking agents include 1,3-dichloropropane, 1,3-dibromopropane, 1,2-dichloropropane, 1,2-dibromopropane, acryloyl chloride, epichlorohydrin, butanediol diglycidyl ether, ethanediol diglycidyl ether, dimethyl succinate, succinyl dichloride, the diglycidal ether of bisphenol A, pyromellitic dianhydride, toluene diisocyanate, ethylene diamine or dimethyl succinate.

A higher level of cross-linking decreases the water-solubility of the polymers, rendering them less absorbable (e.g., by the intestinal tract), and thus substantially limits the activity of the cross-linked polymers to the intestinal tract when they are administered orally or rectally. Because a cross-linked polymer of the invention is non-absorbable, systematic side effects in a patient are largely eliminated. The compositions thus tend to be non-systemic in activity. Typically, the cross-linking agent is present in an amount from about 0.5-35% or about 0.5-25% (such as from about 2.5-20% or about 1-10%) by weight, based upon total weight of monomer plus cross-linking agent.

A preferred cross-linking agent is epichlorohydrin because of its high availability and low cost. Epichlorohydrin is also advantageous because of its low molecular weight and hydrophilic nature, increasing the water-swellability and gel properties of the polyamine.

The molecular weight of polymers of the invention is not believed to be critical, provided that the molecular weight is large enough so that the polymer is non-absorbable by the gastrointestinal tract. Typically, the molecular weight is at least 1,000. For example, the molecular weight can be from about 1,000 to about 5 million, about 1,000 to about 3 million, about 1,000 to about 2 million or about 1,000 to about 1 million.

As discussed above, the polymers can be administered in the form of a pharmaceutically acceptable salt. By “salt” it is meant that the nitrogen group in the repeat unit is protonated to create a positively charged nitrogen atom associated with a negatively charged counterion. The polymers can also include pharmaceutically acceptable salts of acidic and/or basic substituents in the polymers.

Aliphatic amine polymers can be protonated with organic or inorganic acids comprising physiologically acceptable anions. The anions can be partially or completely replaced with other physiologically acceptable anions by various means, including by passing the polymer over an anion exchange resin prior to crosslinking. An aliphatic amine polymer can comprise more than one type of anion.

Examples of suitable anions for aliphatic amine salts include organic ions, inorganic ions or combination thereof, such as halides (Cl⁻ and Br⁻), CH₃OSO₃⁻, HSO₄⁻, SO₄²⁻, HCO₃⁻, CO₃²⁻, nitrate, hydroxide, persulfate, sulfite, acetate, lactate, succinate, propionate, oxalate, butyrate, ascorbate, citrate, dihydrogen citrate, tartrate, taurocholate, glycocholate, cholate, hydrogen citrate, maleate, benzoate, folate, an amino acid derivative, a nucleotide, a lipid, or a phospholipid. Chloride, carbonate and bicarbonate are preferred anions. The counteranions can be the same as or different from each other. For example, the polymer can have two or more different types of couteranions. Divalent and multivalent anions can be counterions to more than one protonated amine.

The aliphatic amine polymers used in the invention which are typically those in which less than 40%, such as less than 30%, particularly less than 20%, and more particularly less than 10%, of the amine groups are protonated.

The aliphatic amine polymer resin can be hydrated. In one example, the resin has a moisture content of about 5% by weight or greater, such as from about 3% to about 10% by weight, and more specifically about 7% by weight for sevelamer (e.g. sevelamer hydrochloride) and from about 8.2% to about 9.2% by weight for colesevelam (e.g. colesevelam hydrochloride). It is to be understood that in embodiments in which the polymer resin is hydrated, the water of hydration is considered to be a component of the resin. Thus, a tablet core of the invention having at least about 95%, at least about 96%, or at least about 98% by weight of a hydrated polymer, includes the water of hydration in the weight of the polymer. Tablet cores can also have at least about 70%, such as at least about 80%, for example, at least about 85%, and more particularly at least about 90% by weight hydrated polymer resin.

An example of a direct compression tablet formulation is described in detail in WO 01/28527 and U.S. Publication No.2002/0054903 A1, the contents of which are incorporated herein by reference in their entireties. For example, the tablet core of the invention can be prepared by a method comprising the steps of: (1) hydrating or drying the aliphatic amine polymer to the desired moisture level; (2) blending the aliphatic amine polymer with excipients; and (3) compressing the blend using conventional tableting technology.

As used herein, an “enteric coating” includes one or more polymeric materials that encase the medicament core. An “enteric coating” is also referred herein as a “topcoat.” An enteric coating of the invention is pharmaceutically acceptable, i.e., non-toxic and does not cause unacceptable side effects at the amounts being administered.

An enteric coating delays the release of a drug and makes it possible for the drug to be released after passage through the stomach, e.g., a particular location within the intestinal tract. The materials from which the enteric coating is prepared and/or the thickness of the enteric coating can be selected to control the period of time before a medicament is released in the intestinal tract (e.g. the small intestine). Specifically, the enteric coating material and/or thickness of the enteric coating can be selected such that a medicament is selectively released in the duodenum (e.g. duodenal bulb, C loop, horizontal portion, and/or ascending portion), jejunum (e.g. proximal jejunum, mid-jejunum and/or terminal jejunum) and/or ileum. For example, it may be advantageous to release the aliphatic amine polymers near the site of entry of the intestinal tract, i.e., duodenum and/or the proximal jejunum. Alternatively, it may be beneficial to have the aliphatic amine polymers released in the terminal jejunum, where most of food absorption has taken place. Also, releasing the aliphatic amine polymers in the mid-jejunum can be advantageous.

Enteric coatings typically comprise polymers with acidic functional groups or polymers with basic functional groups, more typically acidic functional groups when the enteric coating is acid resistant. Enteric coatings of the invention can solubilize in an aqueous solution between about pH 5.0 and about pH 6.0, such as between about pH 5.0 and about 5.5 or between about pH 5.5 and about 6.0. Also, the enteric coatings can solubilize in an aqueous solution between about pH 6.0 and about pH 7.0 at about 37° C., such as between about pH 6.0 and about pH 6.5. Herein, “a pH at which an enteric coating solubilizes” means the minimum pH at which an enteric polymer having acid functional groups or the maximum pH at which an enteric polymer having basic functional groups substantially dissolves (e.g. an enteric coating using a polymer having acid functional groups is largely stable at a pH below the minimum pH, and an enteric coating using a polymer having basic functional groups is largely stable at a pH above the maximum pH).

One type of enteric coating is an acid-resistant coating. An “acid-resistant coating” is resistant to the acidic nature of the stomach, i.e., substantially insoluble at the pH of the stomach (approximately pH 1 to pH 4.5). Acid-resistant coatings typically become soluble at pH values greater than the pH of the stomach, e.g., in the small or large intestine, where the pH gradually increases to neutrality (approximately pH 5.0 to pH 7.2).

A medicament contained inside of an enteric coating becomes available when the enteric coating layer solubilizes and dissolves to a point where rupture occurs. Preferably, release of the medicament, once the enteric coating ruptures, is rapid and complete, e.g., the entire dose is released within about 1-30 minutes, for example, 1-20 minutes, or more specifically 5-10 minutes after the rupture of the enteric coating.

Numerous types of acid-resistant enteric coatings are available. Examples of the acid-resistant coatings include cellulose acetate phthalate, polyvinyl acetate phthalate, shellac, an acrylic acid homopolymer or copolymer, a methacrylic acid homopolymer or copolymer, cellulose acetate trimellitate, hydroxypropyl methylcellulose phthalate or a combination of thereof. A preferred acid-resistant coating material is an acrylic acid homopolymer or copolymer or a methacrylic acid homopolymer or copolymer or a combination thereof. A copolymer of methacrylate and methacrylic acid is particularly preferred. A number of copolymers of methacrylate and methacrylic acid are known in the art and are commercially available. Examples of such polymers are copolymers of methylmethacrylate and methacrylic acid and copolymers of ethylacrylate and methacrylic acid, and sold under the tradename Eudragit® (Röhm GmbH & Co. KG): examples include Eudragit® L100-55, Eudragit® L30-D55, Eudragit® L100, Eudragit® S100-55 and Eudragit® FS 30 D.

Polymer materials which solubilize at pH lower than about 5.5 are particularly suitable for targeting the duodenum, although they can also be used to target other regions of the intestinal tract. Specific examples of such polymers include polyvinyl acetal diethylaminoacetate, as sold under the tradename AEA (Sankyo Co., Ltd.) and hydroxypropylmethylcellulose phathalate, as sold under the tradename HP-50 and HP-55 (Shin-Etsu Chemical Co., Ltd.)

Eudragit® L100-55 and Eudragit® L30-D55 are examples of polymers which are insoluble below about pH 5 and solubilize at pH values greater than about 5.5.

Polymer materials which solubilize at about pH 6.0 or higher are suitable for targeting the jejunum and/or ileum, along with the large intestine. Examples of such polymers include a methylmethacrylate-methacrylic acid (1:1) copolymer (Eudragit® L 100), a methylmethacrylate-methacrylic acid (2:1) copolymer (Eudragit® S 100), an ethylacrylate-methacrylic acid (1:1) copolymer (Eudragit® LD-55), cellulose acetate phthalate, and shellac. Eudragit® S 100, a copolymer of methacrylic acid and methylmethacrylate, having a ratio of free carboxyl groups to ester groups of approximately 1:2 solubilizes at about pH 7.0 or higher, can be used for targeted delivery to the ileum.

These enteric coating materials may be used either individually or as an appropriate mixture thereof. That is, two or more materials can be mixed together in a particular ratio, such that the enteric coating solubilizes at an intermediate pH. For example, mixtures of Eudragit® L 100 and Eudragit® S 100 can allow the release of active ingredients in a pH range from 6.0 to 6.5.

The rupture of an enteric coating and the subsequent release of active ingredients also depends on the amount of coating (i.e., thickness), in addition to the solubility characteristics of the polymer materials of the enteric coating. Because acid-resistant enteric coatings are pH sensitive, they will only solubilize and rupture when exposed to an appropriate environment. Typically, application of a thicker coating will increase the time until rupture of the enteric coating occurs.

In the present application, a tablet or bead core is typically coated with an enteric coating that is about 5% to about 15% of the weight of the tablet core. The amount of enteric coating is typically measured in terms of the weight gain caused by the application of coating layers over the cores. Hence, the amount of enteric coating is expressed relative to the weight of the uncoated tablet or bead core. In one example, a tablet core is coated with an enteric coating that is about 5% to about 7% of the weight of the tablet core. In another example, the enteric coating is about 10% to about 14% of the weight of the tablet core. Examples 3 and 4 demonstrate how the disintegration time of a tablet can be controlled by varying the amount of enteric coating. In these examples, Eudragit L30-D55 was used as an enteric coating, where the amount of the enteric coating applied to the tablet core was about 5% to 15% by weight based on the weight of the tablet core. For this amount of enteric coating, the disintegration time of the tablet at pH 5.75 at 37° C. ranged from about 21 to about 77 minutes (Example 3) and from about 35 to about 98 minutes (Example 4).

Enteric coatings, such as those described above, can be modified by mixing with other known coating products that are not pH sensitive. Examples of such products include copolymers of acrylate and methacrylates with quaternary ammonium groups, sold currently under the tradenames Eudragit® RL and Eudragit® RS and a neutral ester dispersion without any functional groups, sold under the tradenames Eudragit® NE30-D.

An enteric coating can also be a time-release coating. The time-release coatings are degraded away at a relatively constant rate until the coatings dissolve sufficiently for the time-release coatings to rupture. Thus, the time required for the rupture of the enteric coatings is largely time-dependent (i.e., thickness), and largely pH independent. Examples of time-release coating materials include cellulose acetate, cellulose acetate butyrate, cellulose acetate propionate, ethyl cellulose and copolymers of acrylate and methacrylates with quaternary ammonium groups such as Eudragit® RL and Eudragit® RS and Eudragit® NE30-D. The amount of time-release coatings can be selected such that the coating ruptures in about 0.3 to about 10 hours, preferably, about 0.5 to about 4 hours, such as about 0.5 to about 1 hour, about 1 to about 1.5 hours, about 1.5 to about 2 hours, about 2 to about 3 hours and about 3 to about 4 hours. A time-release coating of the invention can be used alone or in combination with an acid-resistant coating.

Tablets and beads of the invention further optionally comprise a water-soluble coating between the enteric coating and the tablet core. The “water-soluble coating” is also referred herein as “sealcoat” or “seal coating”. A tablet with a water-soluble coating of the invention can be prepared by a method comprising the step of contacting a tablet core of the invention described above with a coating solution comprising a solvent, at least one coating agent dissolved or suspended in the solvent and, optionally, one or more plasticizing agents. Preferably, the solvent is an aqueous solvent, such as water or an aqueous buffer, or a mixed aqueous/organic solvent. Preferred coating agents include cellulose derivatives, such as hydroxypropylmethylcellulose, methylcelluose, hydroxylethylcellulose, hydroxyethylmethylcellulose, hydroxylethylethylcelluose and hydroxypropylethylcellulose. Suitable hydroxypropylmethylcellulose (HPMC) solutions include those having HPMC low viscosity and/or HPMC high viscosity. Additional suitable cellulose derivatives include cellulose ethers useful in film coating formulations. Typically, the tablet core is contacted with the coating solution until the weight of the tablet core has increased by an amount ranging from about 0.5% to about 3%, preferably from about 0.5% to about 2.5%, and more preferably from about 0.5% to 1.5%.

An enteric coating layer can be applied over a tablet or bead core with or without a seal coating by conventional coating techniques, such as pan coating or fluid bed coating using solutions of polymers in water or suitable organic solvents or by using aqueous polymer dispersions. Methods of applying enteric coatings can be found in U.S. Pat. Nos. 4,185,088, 5,108,758, 5,681,584, 5,897,910 and 6,200,600, the contents of which are incorporated herein by reference.

The capsules, sachets or papers of the invention can have a first and a second plurality of beads having a different enteric coating or a different amount of enteric coating from each other. In these capsules, sachets or papers, the release of the active ingredient, an aliphatic amine polymer, can be targeted to more than one region of the intestinal tract, such as multiple parts of the small intestine, in a single dosage form. It may be advantageous to use such dosage forms when there are multiple target regions in the intestinal tract, such as to improve efficiency.

Another aspect of the invention relates to a tablet having a tablet core that includes a polymer active ingredient, where the tablet core is coated with a water-soluble coating and the water-soluble coating is coated with an enteric coating. In certain applications, having a water-soluble seal coating and an enteric coating over the seal coating provides for a more consistent disintegration time for a tablet than a tablet having only an enteric coating (there is less variation in disintegration time). The polymer in such tablet cores can be an amine polymer, such as an aliphatic amine polymer (e.g., a water-insoluble aliphatic amine polymer).

Capsules, sachets or papers of the invention can be prepared by conventional techniques known in the art. These capsules, sachets or papers serve as containers for beads having an active ingredient. Soft and hard gelatin capsules are quite common in the art. Polymers that include polyvinyl alcohol, cellulose ethers, polyethylene oxide, starch, polyvinylpyrrolidone, polyacrylamide, polyvinyl methyl ether-maleic anhydride, polymaleic anhydride, styrene maleic anhydride, hydroxyethylcellulose, methylcellulose, polyethylene glycols, carboxymethylcelulose, polyacrylic acid salts, alginates, acrylamide copolymers, guar gum, casein, ethylene-maleic anhydride resin series, polyethyleneimine, ethyl hydroxyethylcellulose, ethyl methylcellulose, hydroxyethyl methylcellulose are known to be used for a sachet. Procedures for manufacturing capsules and sachets are known in the art, for example, the preparation of a water-soluble sachet is disclosed in U.S. Pat. No. 6,632,785, the preparation of a capsule is disclosed in U.S. Pat. No. 4,627,850 and Pharmaceutical Sciences by Remington, Vol. XIV, pp 1671-77, (1970) published by Mack Publishing Co., Easton, Pa., the contents of which as incorporated herein by reference.

A tablet or capsule, sachet or paper of the invention can further comprise one or more excipients, such as plasticizers, hardeners, glidants and lubricants. Excipients included in a tablet can include, for example, colloidal silicon dioxide, diaceylated monoglyceride, stearic acid, magnesium silicate, calcium silicate, sucrose, calcium stearate, glyceryl behenate, magnesium stearate, talc, zinc stearate and sodium stearylfumarate. Excipients included in a capsule can include, for example, colloidal silicon dioxide, lactose, sorbitol and stearic acid. Capsule exteriors can have, for example, titanium dioxide and indigo carmine ink. The excipients can represent, for example, from 0 to about 30% of the tablet core by weight.

The invention further relates to a method for lowering cholesterol in a mammal in need thereof by administering to the mammal a therapeutically effective amount of a tablet, capsule, sachet or paper of the invention described above. A therapeutically effective amount is defined herein as a sufficient amount of an aliphatic amine polymer to treat a mammal in need of lowering serum cholesterol level. For example, a therapeutically effective amount of aliphatic amine polymers is about 0.5 g to about 2 g, such as about 0.5 g to about 1.6 g. Such doses of tablets, capsules, sachets or papers can conveniently be administered to a patient once or twice daily. When administered more than once daily, the therapeutically effective amount can be administered in a series of doses separated by appropriate time intervals such as minutes or hours. The tablets, capsules, sachets or papers described herein can be administered before, with Qr after a meal.

The tablet or capsule, sachet or paper of the invention can be administered alone or in combination with one or more additional pharmaceutical agents. Suitable pharmaceutical agents include, for example, an antihyperlipidemic agent, such as LXR agonists (see WO 01/03705); a plasma HDL-raising agent; an antihypercholesterolemic agent, such as cholesterol biosynthesis inhibitor, for example an HMG-CoA reductase inhibitor (such as a statin), an HMG-CoA synthase inhibitor, a squalene epoxidase inhibitor, or a squalene synthetase inhibitor (also known as squalene synthase inhibitor); an acyl-coenzyme A: cholesterol acyltransferase (ACAT) inhibitor, such as melinamide; probucol; nicotinic acid and the salts thereof and niacinamide; a cholesterol absorption inhibitor such as beta-sitosterol; and LDL (low density lipoprotein) receptor inducer; fibrates such as clofibrate, fenofibrate, and gemfibrizol; vitamin B₆ (also known as pyridoxine) and the pharmaceutically acceptable salts thereof, such as the HCl salt; vitamin B₁₂ (also known as cyanocobalamin); anti-oxidant vitamins, such as vitamins C and E, and beta carotene: a beta-blocker; and angiotensin II antagonist converting enzyme inhibitor; and a platelet aggregation inhibitor, such as fibrinogen receptor antagonists (i.e., glycoprotein IIb/IIIa fibrinogen receptor antagonists) and aspirin.

### EXEMPLIFICATION

### Example 1

### Disintegration Test on 800 mg of Sevelamer HCl Enteric Coated Tablets

Sevelamer hydrochloride tablets were first coated with a seal coat of water soluble polymer (hydroxypropylmethyl cellulose, Table 1). The seal coated tablets were then film-coated in 5 to 15% weight gain range by Eudragit L30D55-containing formulation (Table 2). The site of release of the tablets can be estimated by performing disintegration test in pH 5.75 medium. The data of the disintegration test performed at pH 5.75 clearly showed an increase in disintegration time with an increase in coating level, thereby allowing for determination of coating level desired for release of aliphatic amine polymers, for example, Sevelamer HCl, at desired site in the intestinal tract.

TABLE 1
Component and Composition of Water-Soluble
Coating (Seal Coat)

| Component | Percent Weight (% w/w) | Qty (g/batch) |
|---|---|---|
| Spectrablend SB 50842 | 10 | 200 |
| * DI water | 90 | 1800 |

* DI water was removed during the coating process

TABLE 2
Component and Composition of Enteric Coating (Top Coat)

| | g/batch | | % w/w |
|---|---|---|---|
| Eudragit L30D55 (30% solid) | 2393.0 | g | 59.83 |
| Triethyl citrate | 71.2 | g | 1.78 |
| Glyceryl Monostearate dispersion 10.1%, | 239 | g | 5.98 |
| TEC 10.1% and 0.2% Tween 80 in water | | | |
| (20.4%) (Plas II) | | | |
| * DI water | 1296.8 | g | 32.42 |
| Total | 4000 | g | 100 |

The disintegration test was performed using a dissolution apparatus at 37° C. The enteric coated tablets were exposed to an acidic medium of succinate buffer of pH 4.5 first for 2 hours and then exposed to a succinate buffer of pH 5.75. Rupture time and disintegration time were measured during testing. Rupture time (RT) is the time when the enteric coating of tablets starts to break up, and disintegration time (DT) is the time when a tablet is completely disintegrated.

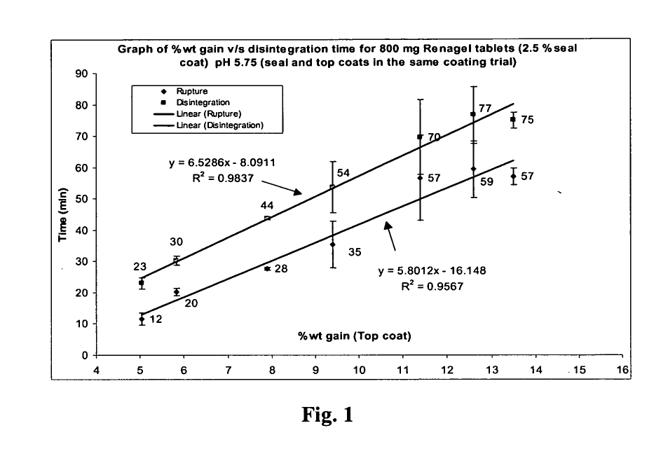

Table 3 summarizes the composition of seal coat and topcoat, disintegration apparatus used, pH of disintegration medium, rupture and disintegration times and testing conditions for 800 mg sevelamer hydrochloride enteric coated tablets. The disintegration times for the tablets with 5.0%, 5.8% and 7.9% weight gain of topcoat and 2.5% of seal coat were 21-25 minutes, 29-32 minutes and 43-44 minutes, respectively. The disintegration times for the tablets with 11.4%, 12.6% and 13.5% weight gains of topcoat and 2.5% of seal coat were 58-82 minutes, 69-87 minutes and 72-77 minutes, respectively (FIG. 1). These results show an excellent control of the site of disintegration through time delay after reaching pH 5.75.

TABLE 3
Disintegration Test on 800 mg of Sevelamer HCl Enteric Coated Tablets

| | Sample 1 | Sample 2 | Sample 3 | Sample 4 |
|---|---|---|---|---|
| Coating Composition: | | | | |
| Seal coat: (Spectrablend) | 2.5% | 2.5% | 2.5% | 2.5% |
| Top-coated (Eudragit L30D55) | 5.0% | 5.8% | 7.9% | 9.4% |
| Testing Conditions: | | | | |
| First 2 hours (acid resistant) | 250 ml of | 250 ml of | 250 ml of | 250 ml of |
| | succinate buffer | succinate buffer | succinate buffer | succinate buffer |
| | (0.05M) pH to 4.5 | (0.05M) pH to 4.5 | (0.05M) pH to 4.5 | (0.05M) pH to 4.5 |
| After 2 hours | 250 ml of | 250 ml of | 250 ml of | 250 ml of |
| | succinate buffer | succinate buffer | succinate buffer | succinate buffer |
| | 0.05 M at pH 5.75 | 0.05 M at pH 5.75 | 0.05 M at pH 5.75 | 0.05 M at pH 5.75 |
| Apparatus: | Dissolution | Dissolution | Dissolution | Dissolution |
| | apparatus III | apparatus III | apparatus III | apparatus III |
| | operated at 17 dpm | operated at 17 dpm | operated at 17 dpm | operated at 17 dpm |
| Number of tablet tested: | N = 4 | N = 3 | N = 4 | N = 3 |
| Results: | | | | |
| Number of tablets disintegrate | 0 | 0 | 0 | 0 |
| in first 2 hours | | | | |
| Rupture Time: | 10 to 14 min | 19 to 21 min | 27 to 28 min | 28 to 43 min |
| Total Disintegration Time: | 21 to 25 min | 29 to 32 min | 43 to 44 min | 45 to 61 min |

| | Sample 5 | Sample 6 | Sample 7 |
|---|---|---|---|
| Coating Composition: | | | |
| Seal coat: (HMPC) | 2.5% | 2.5% | 2.5% |
| Top-coat (Eudragit L30D55) | 11.4% | 12.6% | 13.5% |
| Testing Conditions: | | | |
| First 2 hours (acid resistant) | 250 ml of | 250 ml of | 250 ml of |
| | succinate buffer | succinate buffer | succinate buffer |
| | (0.05M) pH to 4.5 | (0.05M) pH to 4.5 | (0.05M) pH to 4.5 |
| After 2 hours | 250 ml of | 250 ml of | 250 ml of |
| | succinate buffer | succinate buffer | succinate buffer |
| | 0.05 M at pH 5.75 | 0.05 M at pH 5.75 | 0.05 M at pH 5.75 |
| Apparatus: | Dissolution | Dissolution | Dissolution |
| | apparatus III | apparatus III | apparatus III |
| | operated at 17 dpm | operated at 17 dpm | operated at 17 dpm |
| Number of tablets tested: | N = 3 | N = 3 | N = 3 |
| Results: | | | |
| Number of tablets disintegrate | 0 | 0 | 0 |
| in first 2 hours | | | |
| Rupture Time: | 43 to 70 min | 51 to 68 min | 54 to 59 min |
| Total Disintegration Time: | 58 to 82 min | 69 to 87 min | 72 to 77 min |

### Example 2

### Disintegration Test on 800 mg Sevelamer HCl Enteric Coated Tablets with Different Percentage of Seal Coat

Disintegration testing was also performed at 37° C. on tablets with 7.9% and 13.5% weight gain of a topcoat of Eudragit L30D55 and with 1.5% weight gain of a seal coat of Spectrablend SB 50842. The results, as presented in Table 4, indicate that increasing the amount of seal coat from 1.5% to 2.5% does not significantly affect the mean disintegration time, although the variability in disintegration times is reduced.

The tablets with 7.9% topcoat and 2.5% seal coat have a disintegration time of 43 to 44 minutes and the tablets with 7.9% topcoat and 1.5% seal coat have a disintegration time of 34 to 51 minutes. In addition, the tablets with 13.5% topcoat and with 2.5% seal coat have a disintegration time of 73 to 77 minutes and the tablets with 13.5% topcoat and 1.5% seal coat have a disintegration time of 70 to 75 minutes. The results showed that as the percent weight gain of topcoat (Eudragit L30D55) increased, the rupture (RT) and disintegration times (DT) increased.

TABLE 4
Disintegration Test on 800 mg Sevelamer HCl Enteric Coated Tablets with different percent of seal coat

| | Sample 8 | Sample 9 | Sample 10 | Sample 11 |
|---|---|---|---|---|
| Coating Composition: | | | | |
| Seal coat: (Spectrablend) | 1.5% | 2.5% | 1.5% | 2.5% |
| Top-coated (Eudragit L30D55) | 7.9% | 7.9% | 13.5% | 13.5% |
| Testing Conditions: | 250 ml of | 250 ml of | 250 ml of | 250 ml of |
| First 2 hours (acid resistant) | succinate buffer | succinate buffer | succinate buffer | succinate buffer |
| | (0.05M) pH to 4.5 | (0.05M) pH to 4.5 | (0.05M) pH to 4.5 | (0.05M) pH to 4.5 |
| After 2 hours | 250 ml of | 250 ml of | 250 ml of | 250 ml of |
| | succinate buffer | succinate buffer | succinate buffer | succinate buffer |
| | 0.05 M at pH 5.75 | 0.05 M at pH 5.75 | 0.05 M at pH 5.75 | 0.05 M at pH 5.75 |
| Apparatus: | Dissolution | Dissolution | Dissolution | Dissolution |
| | apparatus III | apparatus III | apparatus III | apparatus III |
| | operated at 17 dpm | operated at 17 dpm | operated at 17 dpm | operated at 17 dpm |
| Number of tablets tested: | N = 3 | N = 4 | N = 3 | N = 3 |
| Results: | | | | |
| Number of tablets disintegrate | 0 | 0 | 0 | 0 |
| in first 2 hours | | | | |
| Rupture Time: | 27 to 28 min | 27 to 28 min | 52 to 56 min | 54 to 59 min |
| Total Disintegration Time: | 34 to 51 min | 43 to 44 min | 70 to 75 min | 72 to 77 min |

### Example 3

### Effect of pH of Disintegration Medium on Disintegration Time of Enteric Coated Tablet of Sevelamer HCl, 800 mg

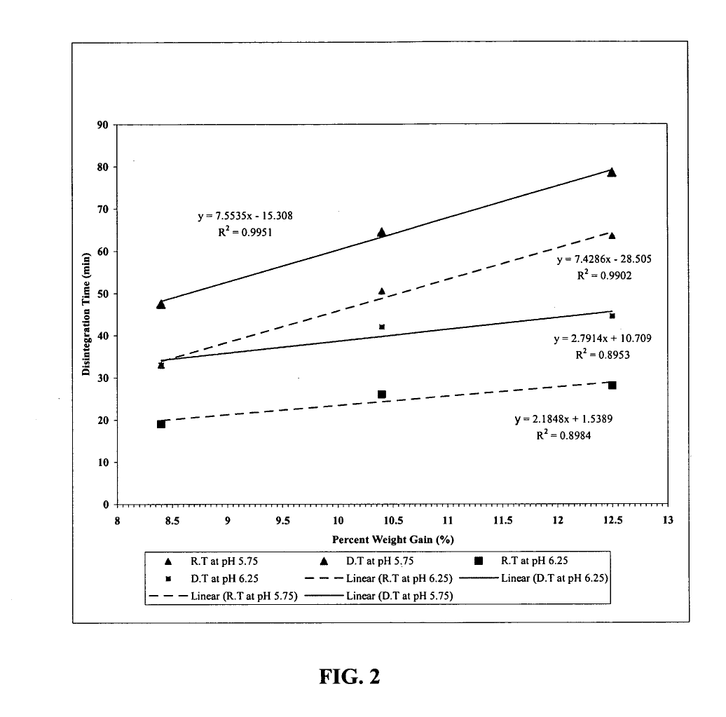

Disintegration testing was performed at 37° C. on 800 mg enteric coated sevelamer HCl tablet using a disintegration medium of 0.05M succinate buffer at pH 6.25 instead of pH 5.75. The composition of seal coat and topcoat, disintegration apparatus used, pH of disintegration medium, rupture and disintegration times and testing conditions are presented in Table 5. The comparative result is shown in FIG. 2, which indicates that the rupture time (RT) and disintegration time (DT) reduce as the pH of disintegration medium increases from pH 5.75 to pH 6.25. This provides an alternate means of ensuring that the delivery system is targeted to the intended release site. If the intestinal tract transit time is faster, the tablet would release sevelamer HCl sooner due to corresponding increase in pH.

TABLE 5
Disintegration Test on 800 mg Sevelamer HCl Enteric Coated Tablet at pH 6.25

| | Sample 1 | Sample 2 | Sample 3 |
|---|---|---|---|
| Coating Composition: | | | |
| Seal Coat (Spectrablend) | 2.5% | 2.7% | 2.4% |
| Top Coat (Eudragit L30D) | 8.4% | 10.4% | 12.5% |
| Testing Conditions: | | | |
| First 2 hours (acid resistant) | 220 ml of | 220 ml of | 220 ml of |
| | succinate buffer | succinate buffer | succinate buffer |
| | (0.05M) pH to 4.5 | (0.05M) pH to 4.5 | (0.05M) pH to 4.5 |
| After 2 hours | 220 ml of | 220 ml of | 220 ml of |
| | succinate buffer | succinate buffer | succinate buffer |
| | 0.05 M at pH 6.25 | 0.05 M at pH 6.25 | 0.05 M at pH 6.25 |
| Apparatus: | Dissolution | Dissolution | Dissolution |
| | apparatus III | apparatus III | apparatus III |
| | operated at 17 dpm | operated at 17 dpm | operated at 17 dpm |
| pH after first 2 hours | 4.5 | 4.5 | 4.5 |
| pH after tablet disintegrated | 8.50 to 8.61 | 8.43 to 8.55 | 8.48 to 8.53 |
| Number of tablet tested: | N = 4 | N = 3 | N = 7 |
| Results: | | | |
| Number of tablets disintegrate | 0 | 0 | 0 |
| in first 2 hours | | | |
| Rupture Time: | 18 to 20 min | 24 to 28 min | 26 to 30 min |
| Total Disintegration Time: | 32 to 34 min | 41 to 43 min | 39 to 50 min |

### Example 4

### Effect of Coating Weight Gain on Disintegration Time

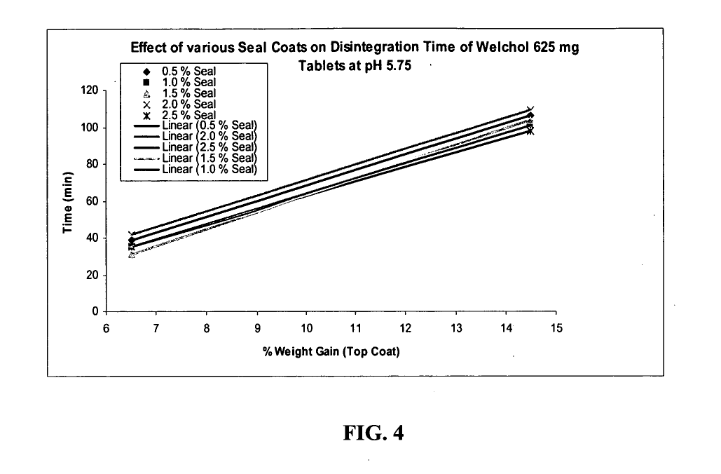

Colesevelam hydrochloride was compressed into tablets and then coated in a similar manner as described in Example 1. The disintegration and rupture test of the tablets were performed at 37° C. Specific test conditions and results are summarized in Table 6. As shown in FIG. 4, the results showed that as percent weight gain of topcoat increased the rupture (RT) and disintegration times (DT) increased.

TABLE 6
Effect of Coating Weight Gain on Disintegration Time

| Lot # | 1 | 2 | 3 | 4 | 5 | 6 |
|---|---|---|---|---|---|---|
| Coating | | | | | | |
| composition | | | | | | |
| Seal Coat (%) | 2.5 | 2.5 | 2.5 | 2.5 | 2.5 | 2.5 |
| Eudragit L30D55 | 6.5 | 8.0 | 10.0 | 11.0 | 13.0 | 14.5 |
| Top Coat (%) | | | | | | |
| Apparatus/Media | | | | | | |
| First 2 hrs | | | | | | |
| 0.05 M Succinate | √ | √ | √ | √ | √ | √ |
| buffer pH 4.5 | | | | | | |
| (250 ml) | | | | | | |
| After 2 hrs | | | | | | |
| 0.05 M Succinate | √ | √ | √ | √ | √ | √ |
| buffer pH | | | | | | |
| 5.75 (250 ml) | | | | | | |
| Apparatus | USP III | USP III | USP III | USP III | USP III | USP III |
| Dips per minute | 17 | 17 | 17 | 17 | 17 | 17 |
| in media | | | | | | |
| #of tablets | 4 | 4 | 3 | 4 | 3 | 3 |
| Tested (n) | | | | | | |
| Results | | | | | | |
| # of coated tablet | 0 | 0 | 0 | 0 | 0 | 0 |
| rupturing in 2 hrs | | | | | | |
| # of coated | 0 | 0 | 0 | 0 | 0 | 0 |
| tablets dis- | | | | | | |
| integrating in | | | | | | |
| 2 hrs | | | | | | |
| After 2 hrs | | | | | | |
| Average Rupture | 25 ± 2.71 | 43 ± 4.99 | 53 ± 1.73 | 67 ± 5.26 | 80 ± 2.65 | 89 ± 11.6 |
| Time (min) | | | | | | |
| Average | 35 ± 1.89 | 50 ± 5.2 | 62 ± 1.53 | 71 ± 4.35 | 88 ± 4.04 | 98 ± 10.6 |
| Time (min) | | | | | | |

While this invention has been particularly shown and described with references to preferred embodiments thereof, it will be understood by those skilled in the art that various changes in form and details may be made therein without departing from the scope of the invention encompassed by the appended claims.

1. A tablet comprising:
a) a tablet core comprising an aliphatic amine polymer or a pharmaceutically acceptable salt thereof; and
b) a pharmaceutically acceptable enteric coating therefor, wherein the enteric coating solubilizes in an aqueous solution between about pH 5.0 and about pH 7.0 at about 37° C.

2. The tablet of claim 1, wherein the enteric coating solubilizes in an aqueous solution between about pH 5.0 and about pH 6.0 at about 37° C.

3. The tablet of claim 1, wherein the enteric coating solubilizes in an aqueous solution between about pH 5.0 and about pH 5.5, or between about pH 5.5 and about pH 6.0 at about 37° C.

4. The tablet of claim 1, wherein the aliphatic amine polymer includes one or more repeat units represented by at least one formula selected from the group consisting of: 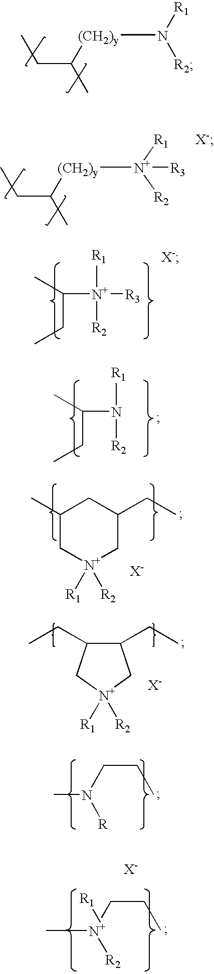
or a salt or a copolymer thereof, wherein:
y is an integer of one or more;
R, R₁, R₂ and R₃, independently, is H, a substituted or unsubstituted alkyl group or an aryl group; and
X⁻ is an exchangeable negatively charged counterion.

5. The tablet of claim 4, wherein the aliphatic amine polymer is cross-linked by means of a multifunctional cross-linking agent.

6. The tablet of claim 5, wherein the aliphatic amine polymer is a polyallylamine.

7. The tablet of claim 6, wherein the polyallylamine is sevelamer.

8. The tablet of claim 7 wherein the sevelamer is sevelamer hydrogen chloride.

9. The tablet of claim 1, wherein the tablet core comprises at least about 70% by weight of the aliphatic amine polymer.

10. The tablet of claim 9, wherein the tablet core comprises at least about 95% by weight of the aliphatic amine polymer.

11. The tablet of claim 1, wherein the enteric coating is an acid-resistant coating.

12. The tablet of claim 11, wherein the acid-resistant coating comprises a polymer selected from the group consisting of cellulose acetate phthalate, polyvinyl acetate phthalate, shellac, an acrylic acid homopolymer or copolymer, a methacrylic acid homopolymer or copolymer, cellulose acetate trimellitate, and hydroxypropyl methylcellulose phthalate or a combination thereof.

13. The tablet of claim 12, wherein the acid-resistant coating comprises a copolymer of methacrylate and methacrylic acid or a combination thereof.

14. The tablet of claim 1, wherein the enteric coating is about 5% to about 15% of the weight of the tablet core.

15. The tablet of claim 14, wherein the enteric coating is about 5% to about 7% of the weight of the tablet core.

16. The tablet of claim 14, wherein the enteric coating is about 10% to about 14% of the weight of the tablet core.

17. The tablet of claim 1, further comprising a water-soluble coating between the enteric coating and the tablet core.

18. The tablet of claim 17 herein the water-soluble coating comprises hydroxypropylmethyl cellulose.

19. The tablet of claim 17, wherein the water-soluble coating is about 0.5% to about 3% of the weight of the tablet core.

20. The tablet of claim 1,
wherein the enteric coating solubilizes in an aqueous solution between about pH 6.0 and about pH 7.0 at about 37° C.

21. The tablet of claim 20, wherein the enteric coating solubilizes in an aqueous solution between about pH 6.0 and about pH 6.5 at about 37° C.

22. The tablet of claim 20, wherein the enteric coating solubilizes in an aqueous solution between about pH 6.5 and about pH 7.0 at about 37° C.

23-38. (canceled)

39. A tablet comprising:
a) a tablet core comprising an aliphatic amine polymer or a pharmaceutically acceptable salt thereof; and
b) a pharmaceutically acceptable enteric coating therefor, wherein the tablet, when orally administered to a mammal, releases the aliphatic amine polymer in the duodenum of the mammal.

40. (canceled)

41. The tablet of claim 39, wherein the aliphatic amine polymer is cross-linked by means of a multifunctional cross-linking agent.

42. The tablet of claim 41, wherein the aliphatic amine polymer is a polyallylamine.

43-46. (canceled)

47. The tablet of claim 39, wherein the enteric coating comprises a copolymer of methacrylate and methacrylic acid or a combination thereof.

48. (canceled)

49. The tablet of claim 39, wherein the enteric coating is about 5% to about 15% of the weight of the tablet core.

50. The tablet of claim 39, further comprising a water-soluble coating between the enteric coating and the tablet core.

51-93. (canceled)
